(19) United States
(12) Patent Application Publication
(10) Pub. No.: US 2014/0128945 A1
(43) Pub. Date: May 8, 2014

(54) NONINVASIVE COOLING DEVICE

(71) Applicant: Heiko Schöning, Hamburg (DE)

(72) Inventor: Heiko Schöning, Hamburg (DE)

(21) Appl. No.: 14/110,602

(22) PCT Filed: Apr. 5, 2012
(86) PCT No.: PCT/EP2012/056360

§ 371 Date: Dec. 11, 2013

(30) Foreign Application Priority Data
Apr. 8, 2011 (DE) 10 2011 001 932.4

(51) Int. Cl. A61F 7/03

(52) U.S. Cl. CPC A61F 7/03
USPC 607/109

(57) ABSTRACT

A device (1) for selectively cooling the brain of a warm-blooded animal, in particular a human being, in a noninvasive manner, includes connectable self-contained cooling elements (4, 18, 19) and fixing elements for fixing in the head/neck area of the warm-blooded animal. The cooling device lowers the brain temperature in warm-blooded animals in a quicker and more effective manner without the need for especially trained medical personnel. This is achieved in that the fixing elements are designed such that the device (1) can be fixed to the warm-blooded animal in order to produce a heat-conductive connection (9, 15, 16) between the cooling elements (4, 18, 19) and at least one carotid artery of the warm-blooded animal.

### CROSS-REFERENCE TO RELATED APPLICATIONS

The present application is a National-Phase Application of International Application No. PCT/EP2012/056360, filed on Apr. 5, 2012, and claims priority to German Patent Application No. 10 2011 001 932.4, filed on Apr. 8, 2011, the content of both of which is herewith expressly included by reference.

### TECHNICAL FIELD OF THE INVENTION

The present invention relates to a device for the noninvasive cooling of the brain of a warm-blooded animal, in particular of a human being, comprising activatable independent cold-producing means and fastening means for fastening in the head/neck region of the warm-blooded animal.

### BACKGROUND OF THE INVENTION

Devices of the type mentioned at the outset are used to treat warm-blooded animals within the context of first aid after occurrence of particular medical conditions, for example ischemic hypoxia of the brain, by intentionally producing localized hypothermia. Unlike other known methods of artificially producing hypothermia, devices of the generic type allow the temperature to be lowered locally in the neck/head region of the warm-blooded animal. They are to be distinguished from methods and devices in which systemic hypothermia, with the additional risks associated therewith, is produced. In particular in the treatment of patients with stroke, cardiac arrest or craniocerebral injury, it is critically important according to medical expertise, in order to minimize reversible damage, that selective hypothermia of the brain should be able to be induced quickly and on the spot, even by medical laypersons.

A device of the type mentioned at the outset is known, for example, from U.S. Pat. No. 4,750,493. The known device is based on a cold-producing means in the form of tablets of ammonium nitrate which, in order to initiate the production of cold, are brought into contact with a water reservoir in order to initiate an endothermic reaction. In this manner it is possible with the known device to produce cold independently, without the need for a connection to an external facility.

The known device is in the form of a hood. This has the disadvantage, therefore, that the desired cooling effect for the brain is obtainable only insufficiently, because cooling takes place through the scalp and the cranium, which have a high heat-insulating effect and constitute a barrier to heat exchange with the brain. This has the disadvantage, therefore, that cooling takes place at least only in a delayed manner, even though cooling as quickly as possible is desirable for the effective prevention of irreversible damage within the context of first aid. Moreover, it is a disadvantage that the temperature in the brain region can sometimes not be lowered sufficiently with the known device because it is applied to the cranium.

The object underlying the present invention” is, therefore, to improve a device of the type mentioned at the outset, while avoiding the disadvantages of the prior art, so that the brain temperature in patients can be lowered more quickly and more effectively even without trained medical personnel.

According to the invention, this object is achieved in a device of the generic type in that the fastening means are so configured that the device can be fastened to the warm-blooded animal in order to establish a heat-conducting connection between the cold-producing means and at least one carotid artery of the warm-blooded animal. In human patients in particular, the carotid arteries are disposed in the neck region comparatively close to the skin surface. They act, so to speak, as heat conductors for the brain. Because it is provided according to the invention, by suitably configuring the fastening means, to fasten the cold-producing means to produce a heat-conducting connection with at least one carotid artery of the warm-blooded animal, selective cooling of the brain can therefore advantageously be achieved quickly and with minimal heat losses and side effects.

If, in an advantageous embodiment of the invention, the fastening means are so configured that the device can be fastened such that it substantially covers with the cold-producing means at least one region that is in heat exchange with a carotid artery of the warm-blooded animal, targeted cooling of the blood flowing to the brain can advantageously be achieved by direct heat exchange.

In a preferred embodiment of the invention, the cold-producing means comprise means for initiating an endothermic reaction, in particular by bringing a first reagent together with a second reagent. This configuration of the cold-producing means advantageously allows cold production independently of external energy sources or the like. In addition, cold production is advantageously possible without problems even by medical laypersons, for example, in first-aid situations. To that end it is simply necessary in particular for the first reagent to be brought into contact with the second reagent, for example by opening a water-containing container for mixing with a salt. As the salt dissolves, the mixture absorbs ambient heat, as is known per se.

For example, ammonium nitrate (NH₄NO₃), can be brought into contact with water in order to obtain the cooling action according to the invention. Likewise, other salts can be dissolved in water in order to achieve this effect. For example, urea, sodium chloride, potassium chloride or magnesium chloride are suitable as the reagent that is to be dissolved in water.

In a preferred embodiment of the invention, the cold-producing means comprise a flexible tubular container containing a first flexible container filled with the first reagent and a second flexible container, chemically sealed with respect thereto, filled with the second reagent. Within the context of the invention, the second container can in particular be a plastic bag filled with water. Likewise, the first, outer container can be configured as a plastic container which contains, in addition to the first reagent, the water-filled plastic sheath. Within the context of the invention, films of polyamide, polyethylene or polypropylene, or mixtures thereof, have been found to be suitable materials for the containers. When choosing the material it is important according to the invention that chemical separation of the reagents stored in the two containers is ensured even over long periods of time. On the other hand, it should be possible to burst the water-containing bag in order to start the endothermic reaction.

If, in a further development of the invention, the first container is divided into two or more compartments that are in communication with one another, the first reagent being distributed over the compartments, the progress of the cooling action over time can advantageously be controlled. To that end it is, for example, possible within the context of the invention for a different amount of the first reagent to be contained in the different compartments, in order to achieve a delayed onset of the endothermic reaction after the second reagent, normally water, has reached the respective compartment. It is likewise possible within the context of the invention to dispose different reagents, such as different salts, in the different compartments in order to produce a uniform cooling capacity that is as long-lasting as possible.

In particular, according to the invention, the first reagent can advantageously be configured to react endothermically with the second reagent, the first reagent being present in the form of a granulate having a large number of grains, in the form of a suspension and/or in the form of a gel.

If, moreover, in an embodiment of the invention, at least some of the grains are provided with a coating that retards and/or accelerates the endothermic reaction with the second reagent, a longer-lasting, uniform cooling action for the brain can advantageously be achieved. However, according to the invention, this is advantageously not obtained at the expense of the quick provision of an initial cooling capacity that is critical for preventing irreversible damage in warm-blooded animals. Within the context of the invention, a coating for acceleration can in particular comprise a catalyst.

According to the invention, the retarding coating of the grains can in particular comprise cellulose.

In a particular embodiment of the invention, a uniform cooling action that begins within a short period of time and at the same time is long-lasting can be achieved if the granulate consists of a mixture of grains having different volumes.

The same effect is achieved by another advantageous embodiment of the invention, according to which the granulate consists of a mixture of grains having coatings of different substances and/or of grain shaving different layer thicknesses. Within the context of his specialist knowledge, the appropriate skilled person will be able to determine suitable mixture compositions in respect of layer thickness and coating materials by means of routine tests, in order to produce a desired profile of the cooling capacity over time.

In order advantageously to achieve the fastening according to the invention of the device to the patient for establishing a heat-conducting connection between the cold-producing means and a carotid artery, it is provided in an embodiment of the invention that the fastening means are in the form of a collar for fastening around the neck of the warm-blooded animal. In particular, Velcro fasteners or the like can be provided for closing the collar.

The device is further improved if as pressure fastening in tape form is additionally attached around the preferably collar-like device for tightening, in order to optimize the application pressure of the device in the region of the carotid arteries.

If the pressure closure intake form is widened by material reinforcements solely in the region of the carotid arteries, the application pressure in that region is additionally optimized as compared with the remainder of the tape.

In another advantageous embodiment of the invention, this is achieved in that the fastening means are in the form of a life jacket for draping around the neck of the warm-blooded animal. According to the invention, it must be ensured that the cold-producing means are in contact with regions of the neck of the warm-blooded animal that are in heat-conducting connection with the carotid arteries.

The device according to the invention is further improved if heat-conducting means, in particular comprising a gel, preferably a hydrophilic gel, for contacting a skin region of the warm-blooded animal are arranged in the vicinity of at least one carotid artery, in order to assist heat transfer via the heat-conducting connection. In this connection, a mixture of water with methylparaben (4-hydroxybenzoic acid methyl ester) in particular has been found to be suitable for achieving heat conduction that is as effective as possible between the carotid arteries and the cooling means. Moreover, it is expedient according to the invention to provide the heat-conducting means substantially only in the immediate vicinity of the carotid arteries of the warm-blooded animal, in order to focus the cooling capacity in a targeted and selective manner on the carotid arteries.

In this connection, the heat-conducting means can in particular comprise according to the invention a pad-like carrier, which is preferably provided with a protective film which can be removed for the designated Fuse. The usability of the device according to the invention as a first-aid measure by medical laypersons, for example in patients following cardiac arrest and resuscitation, is thereby advantageously assisted because, in order to use the device, it is simply necessary to start the endothermic reaction, for example by squeezing and bursting a water bag, and to remove the protective film from the heat-conducting means before the device is applied to the neck of the warm-blooded animal. The device is thus easier to handle than, for example, when a cooling gel or the like provided in a separate tube has to be applied as the heat-conducting means. I

In a further advantageous embodiment of the invention, temperature-regulating means are provided in order to counteract cooling by the cold-producing means that falls below a specified temperature value. In particular, when the device according to the invention is used as a first-aid measure by medical laypersons, it is thus possible to counteract the occurrence of excessive cooling of the brain, which would endanger the warm-blooded animal. In particular, the configuration of the device can be set at the target temperature range of from 32° C. to 34° C. in the brain, that is to say the range for mild therapeutic hypothermia.

Suitable temperature-regulating means are in particular passive regulators.

In particular, in a preferred embodiment of the invention, the temperature-regulating means can comprise latent heat storage means, preferably paraffin-like substances. Within the context of the present invention, latent heat storage is understood as being the storage of heat in a material that undergoes a phase transition. So-called phase-change materials (PCM) are particularly suitable as latent heat storage means.

If a PCM is used according to the invention as the temperature-regulating means, that material can in particular be present in the form of microcapsules.

Into which paraffin-like substances are incorporated, in a manner known per se, a storage of heat takes place in the case of the phase transition from solid to liquid when the material absorbs heat and thus keeps the temperature downwardly stable. On the other hand, a phase transition from liquid to solid occurs, as is known, when the material gives off heat and thus keeps the temperature upwardly constant.

In particular, in an embodiment of the invention, the temperature-regulating means can be arranged to inhibit a heat-conducting connection between the cold-producing means and the warm-blooded animal in regions that are substantially not in heat exchange with a carotid artery of the warm-blooded animal. Advantageously, this arrangement according to the invention of the temperature-regulating means has the effect that the transfer of the cooling capacity from the cold-producing means to the carotid arteries is not impaired by the temperature-regulating means, it being ensured at the same time that the region outside the regions that are in heat exchange with the carotid arteries is not cooled in an undesirable manner. When choosing the temperature-regulating means, in particular if they are configured as latent heat storage means, a material having a phase transition at the temperature that is to be set will be selected, in a manner known tithe person skilled in the art.

If indicator means are provided for indicating that the cold-producing means are activated, it is easier for the operator, who will frequently be a medical layperson, to determine whether he has already, activated the cold-producing means or not. For example, a light-emitting diode can be used as the indicator means, which is switched on at the same time as the cold-producing means are activated.

In a development of the invention, temperature-indicating means can further be provided. In the simplest case within the context of the invention, a color-change field can be used, which is attached tithe device in such a manner that it measures a temperature of the warm-blooded animal. In this connection it is described, for example in JP 09313520 A, that the core temperature of the warm-blooded animal can be inferred by measuring the temperature on the skin. When using the device according to the invention, this can be an important parameter for indicating whether, in an undesired manner, systemic hypothermia is present, in which case the treatment should be terminated.

In a preferred embodiment of the invention, the device is composed substantially like a sandwich in layers, a cooling layer containing the cold-producing means being substantially covered by a separating layer which serves to insulate the cooling layer from the warm-blooded animal, the separating layer having openings for establishing the heat-conducting connection.

According to a development, the device according to the invention can be provided with a moisture-absorbent, in-particular superabsorbent, coating. Advantageously, when the device according to the invention is used on a warm-blooded animal, the superabsorbent material can be wetted in order to achieve long-lasting cooling by evaporative cooling on the skin of the warm-blooded animal. The superabsorbent materials can additionally absorb sweat of the warm-blooded animal, in order to enhance this action.

The invention is described by way of example by means of a preferred embodiment with reference to a drawing, further advantageous details becoming apparent from the figures of the drawing.

Parts that are the same in terms of function have been given the same reference numerals.

### BRIEF DESCRIPTION OF THE DRAWINGS

In detail, in the figures of the drawing:

FIG. 1 shows a schematic section through a device according to the invention for cooling the brain;

FIG. 2 shows a top view of the device according to the invention according to FIG. 1 on the side that is to face the patient, viewed in the direction of arrow II according to FIG. 1;

FIG. 3 shows a view of the side of the device according to FIG. 1 that is to be remote from the patient, viewed in the direction of arrow III in FIG. 1;

FIG. 4 shows a section through the device according to FIG. 1 along line IV-IV in FIG. 1;

FIG. 5 shows a section through the device according to the invention according to FIG. 1 along line V-V in FIG. 1;

FIG. 6 shows a top view corresponding to the representation in FIG. 2 of an alternative embodiment of a device according to the invention.

### DETAILED DESCRIPTION OF THE DRAWINGS

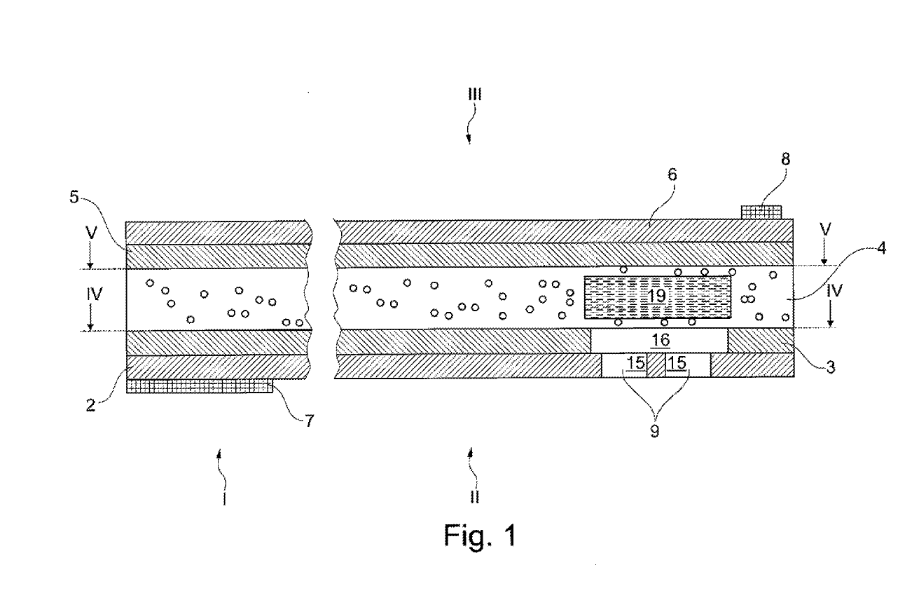

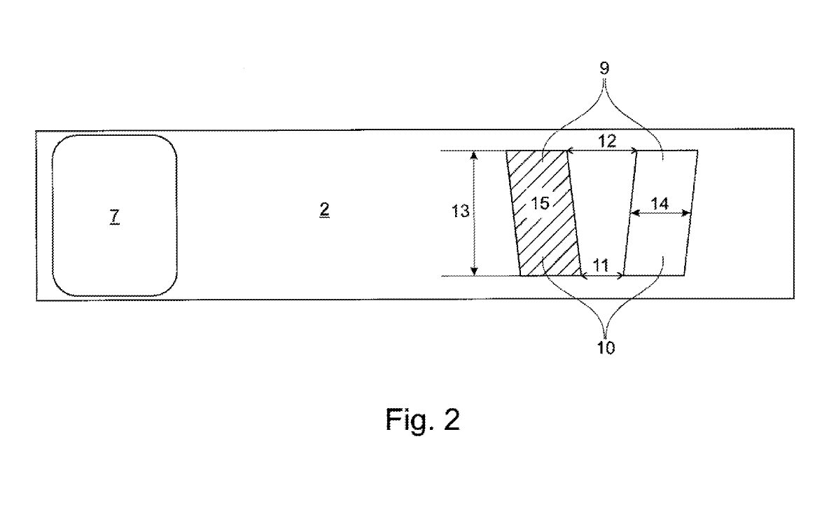

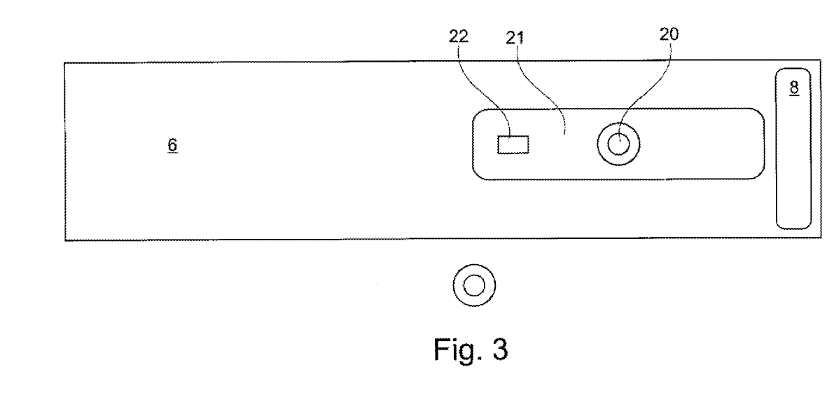

FIG. 1 shows, in a schematic sectional view, a brain cooler 1 as an embodiment of a device according to the invention for the noninvasive, selective cooling of the brain of a patient. As can be seen in the top views according to FIGS. 2 and 3, the brain cooler 1 is generally in the form of a wide rectangular band which, like a collar, can be placed around the neck of a human patient. The brain cooler 1 is composed in several layers like a sandwich.

A first separating layer 2 is provided for direct application to the neck of the patient, in the case of the designated use of the brain cooler 1. Adjoining the separating layer 2 is a temperature-stabilizing layer 3. Adjoining the temperature-stabilizing layer 3, in the direction of the side of the brain cooler 1 that is remote from the patient, is a cooling layer 4. The cooling layer 4 is covered on the side that is to be remote from the patient with a further temperature-stabilizing layer 5. Finally, adjoining the temperature-stabilizing outside layer 5, again on the side remote from the patient, is a highly insulating outside layer 6. The structure of the individual layers and the function thereof will be described below with reference to FIGS. 2 to 5.

By means of FIG. 1, it is shown that the brain cooler 1, which as a whole is flexible, is provided, on the side of the separating layer 2 that is remote from the patient, with a soft Velcro pad. The soft Velcro pad 7 serves to close the brain cooler 1 in the manner of a ring by means of a hard Velcro pad 8 provided on the side of the outside layer 6 that is remote from the patient. In order to apply the brain cooler 1 around the neck of a patient, the brain cooler 1 is placed with the separating layer 2 on the neck of the patient and is guided around the neck. Finally, the hard Velcro pad 8 is guided beneath the region covered by the soft Velcro pad 7 and closed with the soft Velcro pad 7. Alternatively, any other closing method, in particular also a magnetic closure or hooks or the like, can be used within the context of the invention. The separating layer 2 consists of neoprene, in particular having a thickness of 1.5 or 2 mm.

As can be seen in the sectional view according to FIG. 1, the separating layer 2 is interrupted by two openings 9 arranged as a pair. Accordingly, there is a direct heat-conducting connection in the region of the openings 9 between the outside of the separating layer 2 that is to face the patient and deeper-lying layers of the brain cooler 1.

FIG. 2 shows a top view, in the direction of arrow II according to FIG. 1, of the side of the separating layer 2 that is to face the patient. In FIG. 2, the configuration of the openings 9 can clearly be seen. The openings 9 each have the shape of a parallelogram wherein, starting from a mutually parallel lower side 10, they move apart from one another in a V shape. A distance 11 between the two openings 9 is therefore about 2 mm at the lower sides 10, whereas a distance 12 at the upper sides of the openings 9, which are likewise parallel, is 40 mm. The height 13 of the openings 9 is about 80 mm. The width 14 of the openings 9 is about 30 mm. This configuration and arrangement of the openings 9 in the separating layer 2 allows the brain cooler 1 to be placed around the neck of a human patient in such a manner that the openings 9 are arranged above regions of the neck in which the carotid arteries extend close to the surface and thus permit heat exchange with the surroundings. The openings 9 in the separating layer 2 are in particular filled with a cooling plaster 15 impregnated with contact gel. The cooling plaster 15 is preferably impregnated with hydroxyethyl cellulose gel and/or non-ionic-water-containing liniment and optionally gold-containing nanoparticles and is provided with a removable protective film on the side that is to face the patient. The cooling plaster 15 is shown schematically by hatching in FIG. 2 only for the opening 9. However, according to a preferred embodiment of the invention, both openings 9 can in fact be filled with a cooling plaster containing cooling or contact gel.

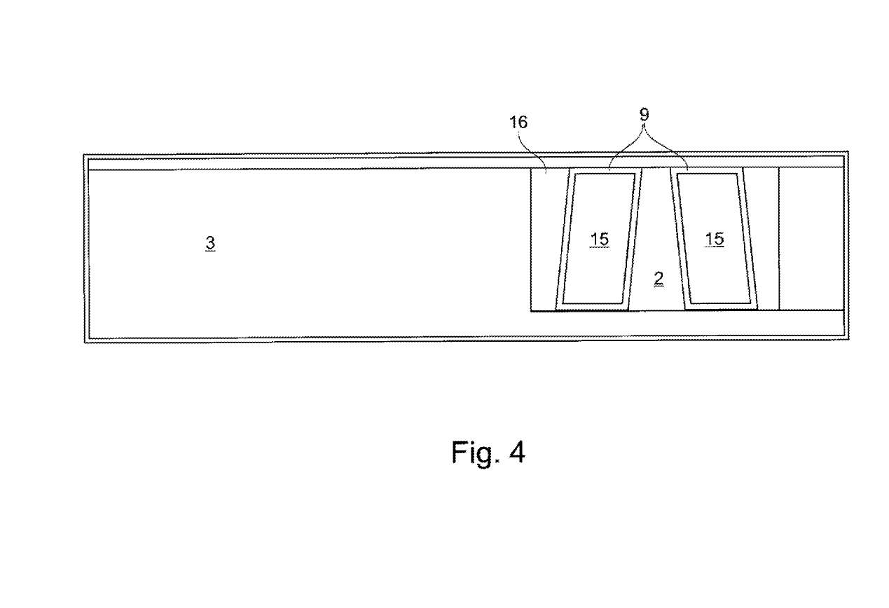

As can be seen particularly well in FIG. 4 in conjunction with the sectional view according to FIG. 1, FIG. 4 being a sectional view along line IV-IV according to FIG. 1, the temperature-stabilizing layer 3 which adjoins the separating layer 2 is interrupted by a rectangular opening 16 in the region of the openings 9 in the separating layer 2. The height of the opening in the temperature-stabilizing layer 3 corresponds substantially to the height 13 of the openings 9 in the separating layer 2.

The temperature-stabilizing layer 3 consists of a latent heat storage material of the thermal nonwoven type. Within the context of the invention, a multicomponent fiber based on paraffin hydrocarbons has been found to be particularly suitable as the latent heat storage material. Such a material and its particular structure to achieve the temperature-stabilizing properties is described, for example, in DE 601 24 275 T2, to the disclosure of which reference is expressly made. The appropriate skilled person will also find a material configuration based on microencapsulated phase-change materials suitable for use as the temperature-stabilizing layer 3 within the context of the present invention in DE 699 23 566 T2. The heat-insulating article described in DE 698 23 690 T2, in which a phase-change material contained in a plurality of microspheres contains paraffin hydrocarbons, is also suitable for the temperature-stabilizing layer 3 according to the invention.

According to the invention, the material of the temperature-stabilizing layer 3 is preferably chosen for setting at “a. temperature value of 11.5° C. Accordingly, the melting point of the phase-change material is to be chosen substantially at 11.5° C. This can be achieved by the person skilled in the art within the context of routine tests by finding suitable mixtures, for example of paraffin hydrocarbons with different chain lengths.

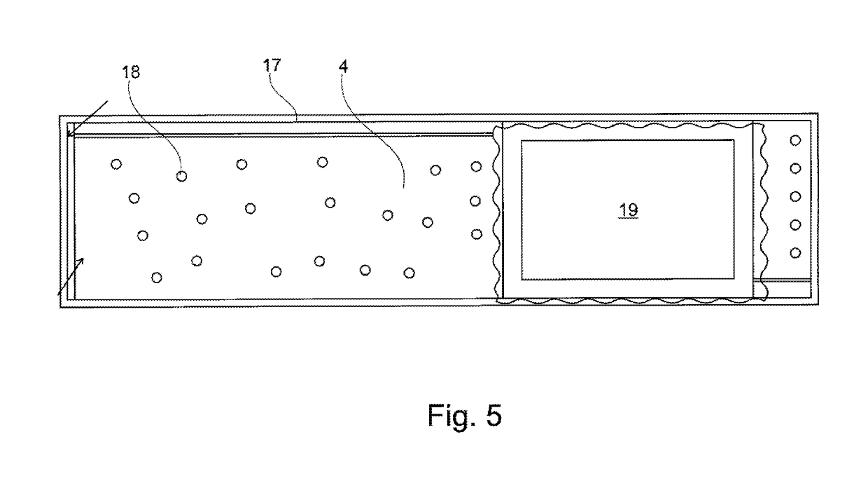

As can be seen particularly well in FIG.” 5 in conjunction with FIG. 1, a cooling layer 4 adjoins the temperature-stabilizing layer 3. The representation according to FIG. 5 corresponds to a section along line V-V in FIG. 1, a part-section of the separating layer 2 additionally being shown cut open. As can be seen in FIGS. 1 and 5, the cooling layer consists of an outer film tube 17. In the outer film tube 17, which according to the embodiment described here is produced in particular of a mixture of polyamide and polyethylene, there is a plurality of grains 18 of ammonium nitrate and/or urea. The grains 18 are distributed inside the inner volume of the outer film tube 17. The outer film tube 17 further contains a water bag 19 made of a polyamide/polyethylene mixture. The water bag 19 is completely closed in order initially to keep the water contained therein completely separate from the grains 18 in the film tube 17. The outer film tube 17 is likewise closed in a fully sealed manner, so that neither the grains 18 nor the water bag 19 nor liquid water can escape from the film tube 17. According to the embodiment described here, the film tube 17 forming the cooling layer 4 extends over the entire extent of the brain cooler 1.

As can again be seen particularly well in FIG. 1, a further temperature-stabilizing layer 5 adjoins the cooling layer 4 on the side of the brain cooler 1 that is to be remote from the patient. With regard tithe material and the other properties, with the exception of the melting point, which in this case is −1.5° C., the further temperature-stabilizing layer 5 is of substantially the same construction as the temperature-stabilizing layer 3 described above, which adjoins the separating layer 2, which is brought into contact with the patient; However, unlike the temperature-stabilizing layer 3 between the separating layer 29 and the cooling layer 4, the temperature-stabilizing layer 5 between the cooling layer 4 and the outside layer 6 is not interrupted by an opening. 1

Finally, FIG. 3 shows a top view of the brain cooler 1 corresponding to arrow III in FIG. 1, showing the outside layer 6. The Outside layer 6 consists of neoprene having a thickness of preferably 1.5 or 2 mm. As can be seen in FIG. 3, a pressure point 20 is made visible to the user on the surface of the outside layer 6 by colored marking. The pressure point 20 is positioned on the outside layer 6 in such a manner that, in the state in which the brain cooler 1 is applied to the patient, the pressure point 20 is arranged on the neck of the patient approximately above the larynx. With regard to the cooling layer 4, the pressure-point 20 is marked so that it is located approximately over the middle of the water bag 19, which in turn is arranged inside the outer film tube 17 substantially in the region of the opening 16 inside the temperature-stabilizing layer 3 and accordingly above the openings 9 in the separating layer 2.

It can further be seen in FIG. 3 that a substantially rectangular LED light strip 21 is arranged around the pressure point 20. This is fixed to the surface of the neoprene outside layer 6 by adhesive bonding, for example. The LED light strip 21 is of commercial form. For supplying electrical power to the LED light strip 21, a cutout 22 is incorporated into the outside layer 6, through which a cable of the LED light strip 21 is guided to the battery and switch electronics (not shown in the figure) located beneath. The switch electronics of the LED light strip 21 is so configured and arranged that, when pressure is applied to the pressure point 20, the LED light strip 21 is switched on above a minimum pressure in order to indicate that the pressure point 20 has been activated and the water bag. 19 has been burst in order to start the endothermic reaction within the cooling layer 4.

The individual layers and components of the brain cooler 1 are connected together by stitching or adhesive bonding. In particular, the cooling plasters 15 can be stitched to the separating layer 2 at the edges of the openings 9. Furthermore, the temperature-stabilizing layer 3 is preferably stitched to the separating layer 2 or integrated therein by high-pressure sputtering. Likewise, the outer film tube 17 is stitched to the separating layer 2 at film seams surrounding it, which are not shown in the figures. The second temperature-stabilizing layer 5, which adjoins the cooling layer 4, is further stitched or adhesively bonded to the separating layer 2. Finally, the separating layer 2 is stitched to the outside layer 6. The stitching is not visible in the figures, in particular in FIG. 1.

In order to use the brain cooler 1 according to the invention according to FIGS. 1 to 5 for the immediate treatment of a patient with, for example, a stroke within the context of first aid, the procedure described below is followed. The user places the brain cooler 1 on a firm surface with the outside layer 6 upwards. He then presses the pressure point 20 with his thumb until the LED light strip 21 illuminates. Pressure is thereby exerted on the water bag 119 arranged beneath the cooling layer 4, which pressure causes the water bag 19 to burst. This leads to mixing of the water contained in the water bag 19 with the grains 18 located inside the outer film tube 17. The grains 18 then dissolve in the mixture within the context of an endothermic reaction with the absorption of heat. This heat is taken from the carotid arteries of the patient via the heat-conducting connection through the opening 16 and the two cooling plasters 15 contained in the openings 9 in the separating layer 2. The blood supplied to the brain of the patient is thereby cooled in a targeted manner in order to produce selective hypothermia in a controlled manner only in the brain. The amount of energy is determined so that significant systemic hypothermia does not occur.

The separating layer 2, and in particular the temperature-stabilizing layers 3 and 5 surrounding the cooling layer 4, ensure that, on the one hand, heat exchange takes place substantially only via the mentioned heat conducting channel through the openings 16, 9 and the cooling plaster 15, in order selectively to cool only the carotid arteries of the patient. The temperature-stabilizing layers 3, 5 also counteract undesired cooling of the brain of the patient below a temperature value which, in this example, is chosen as 32° C.

In the production of the brain cooler 1, the development of the cooling capacity within the cooling layer 4 can be controlled in a targeted manner if the size of the grains 18 is varied and/or if a specific size distribution of the grains 18 is used. It is also possible for all or some of the grains 18 to be provided with a coating that retards the endothermic reaction, for example a coating of cellulose. The outer film tube 17 can be divided into compartments, which is not shown in the figures. In particular, a compartment can be so defined that the water bag 19 is prevented from slipping relative to the pressure point 20 on the outside layer 6.

In this manner, a brain cooler is proposed according to the invention as a preferred embodiment of a device for the noninvasive, selective cooling of the brain according to the preamble of claim 1, which can be used on the spot as a first-aid measure by medical laypersons for immediate treatment in particular with mild therapeutic hypothermia. Advantageously, targeted cooling only of the brain takes place by action on the carotid arteries. The particular material composition and arrangement of the brain cooler according to the invention additionally ensure that excessive cooling, which is medically harmful, is prevented.

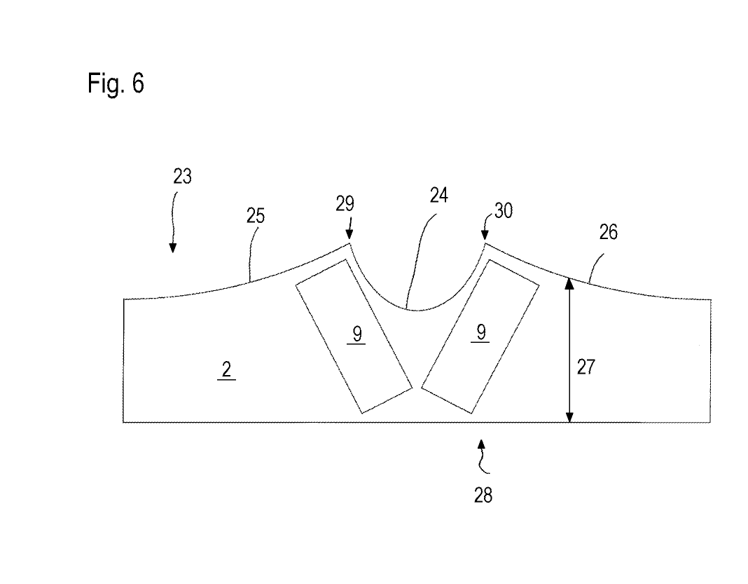

FIG. 6 shows a top view corresponding to the representation shown in FIG. 2 of an alternative embodiment of a device according to the invention for cooling the brain. The inner structure and basic mode of operation of the device showman FIG. 6 can correspond to the structure and basic mode of operation of the device described in relation to FIGS. 1 to 5.

However, the embodiment shown in FIG. 6 has a particularly advantageous form of a top edge 23 of the device. This has, in a region between the openings 9 arranged in a V shape, a concave portion 24, the curvature of which is so chosen that the device is suitable for establishing a particularly good heat-conducting connection between the carotid arteries and the openings 9 in view of the shape of the body in the transition region between the neck and the jawbone.

A further concavely curved portion 25, 26 in each case adjoins the concave portion 24 of the top edge 23 on both sides. The concavely curved portions 25, 26 have a smaller curvature than the concave portion 24 between the openings 9 in the top edge 23 of the brain cooler 1, so that they match the curvature of the periphery of the neck of the patient when the brain cooler is applied to the neck of a patient.

Moreover, the curvature of adjacent portions 24, 24 or of adjacent portions 24, 26 is such that a distance 27 of the top edge 23 from the straight bottom edge 28 decreases as the distance from the concave portion 24 increases. As a result, the concave portion 24 in the region between the openings 9 is delimited on both sides by tapering regions 29, 30. When a brain cooler according to FIG. 6 is applied to the neck, for example with the aid of a Velcro fastener or other devices known per se, it adapts particularly well to the anatomical shape of a human neck owing to the shape of the top edge 23 shown in FIG. 6 with the concave portions 24, 25, 26 and is thus able to establish a particularly good heat-conducting connection with the carotid arteries in the region of the openings 9.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. In particular, elements or features of one embodiment may be may be combined with or replace elements or features of a different embodiment. All such modifications and variations are within the scope of the invention as determined by the appended claims when interpreted in accordance with the breadth to which they are fairly, legally, and equitably entitled.

1. A device (1) for non-invasive cooling of a brain of a warm-blooded animal, comprising activatable independent cold-producing elements (4, 18, 19) and a fastening arrangement for fastening in the head/neck region of the warm-blooded animal, wherein the fastening arrangement is so configured that fastening the device (1) to the warm-blooded animal establishes a heat-conducting connection (9, 15, 16) between the cold-producing elements (4, 18, 19) and at least one carotid artery of the warm-blooded animal.

2. The device (1) according to claim 1, wherein the fastening arrangement is so configured that the cold-producing elements (4, 18, 19) after fastening substantially cover at least one region that is in heat exchange with a carotid artery of the warm-blooded animal.

3. The device (1) according to claim 1, wherein the cold-producing elements (4, 18, 1.9) are configured for initiating an endothermic reaction.

4. The device (1) according to claim 1, wherein the cold-producing elements (4, 18, 19) comprise a flexible, tubular container (17) containing a first flexible container filled with a first reagent (18) and a second flexible container (19) filled with a second reagent, the second flexible container being chemically sealed off from the first flexible container.

5. The device (1) according to claim 4, wherein the first flexible container (17) is divided into two or more compartments that are in communication with one another, the first reagent (18) being distributed over the compartments.

6. The device (1) according to claim 4, wherein the first reagent (18) is configured to react endothermically with the second reagent, the first reagent (18) being in the form of a granulate with a large number of grains, in the form of a suspension or gel.

7. The device (1) according to claim 6, wherein at least some of the grains are provided with a coating that changes a reaction speed of the endothermic reaction with the second reagent.

8. The device (1) according to claim 7, wherein the coating is a retarding coating and comprises cellulose.

9. The device (1) according to claim 6, wherein the granulate consists of a mixture of grains having different volumes.

10. The device (1) according to any of claim 6, wherein the granulate consists of a mixture of grains (18) having coatings that differ in at least one of materials and thicknesses.

11. The device (1) according to claim 1, wherein the fastening arrangement is in the form of a collar for fastening around the neck of the warm-blooded animal.

12. The device (1) according to claim 1, wherein the fastening arrangement is in the form of a life jacket for draping around the neck of the warm-blooded animal.

13. The device (1) according to claim 1, wherein the heat-conducting connection (15) includes at least one of a gel and a liniment configured for covering a skin region of the warm-blooded animal in the vicinity of at least one carotid artery, in order to assist heat transfer via the heat-conducting connection.

14. The device (1) according to claim 1, wherein the heat-conducting connection comprises a pad-like carrier (15).

15. The device (1) according to claim 1, further comprising temperature-regulating layers (3, 5) for counteracting cooling below a given temperature value, caused by the cold-producing elements (4, 18, 19).

16. The device (1) according to claim 15, wherein the temperature-regulating layers (3, 5) comprise latent-heat storage material.

17. The device (1) according to claim 15, wherein the temperature-regulating layers (3, 5) are arranged to inhibit a heat-conducting connection between the cold-producing elements (4, 18, 19) and the warm-blooded animal in regions that are substantially not in heat exchange with a carotid artery of the warm-blooded animal.

18. The device (1) according to claim 1, further comprising indicators (21) configured to indicate that the cold-producing elements (4, 18, 19) are activated.

19. The device (1) according to claim 1, further comprising temperature indicators.

20. The device (1) according to claim 1, wherein the device comprises layers (2, 3, 4, 5, 6) including a cooling layer (4) containing the cold-producing elements. (4, 18, 19) and substantially covered by a separating layer (2) for heat insulation of the cooling layer from the warm-blooded animal, the separating layer (2) having openings (9) for establishing the heat-conducting connection.
